(19) United States
(12) Patent Application Publication
(10) Pub. No.: US 2006/0177870 A1
(43) Pub. Date: Aug. 10, 2006

(54) Immunoassays

(75) Inventors: Yip Tai-Tung, Cupertino, CA (US); Eric Thomas Fung, Mountain View, CA (US)

Correspondence Address: WOODCOCK WASHBURN LLP, ONE LIBERTY PLACE, 46TH FLOOR, PHILADELPHIA, PA, 19103, US

(73) Assignee: Ciphergen Biosystems, Inc, Fremont, CA (US)

(21) Appl. No.: 10/555,078

(22) PCT Filed: Apr. 16, 2004
(86) PCT No.: PCT/US04/11843

§ 371 Date: Apr. 5, 2006

Related U.S. Application Data
(60) Provisional application No. 60/477,358, filed on Jun. 11, 2003.
(60) Provisional application No. 60/536,913, filed on Jan. 16, 2004.

(51) Int. Cl. G01N 33/53

(52) U.S. Cl. 435/7.1

(57) ABSTRACT

Methods for discriminating between related proteins and other analytes in a sample and for correlating protein expression with disease states.

### CROSS REFERENCE TO RELATED APPLICATIONS

This application claims priority to U.S. Provisional Application Ser. No. 60/466,358 filed Apr. 28, 2003 and U.S. Provisional Application Ser. No. 60/536,913 filed Jan. 16, 2004, each which is incorporated herein by reference in its entirety.

### FIELD

This invention relates to the fields of protein biochemistry and clinical diagnostics.

### BACKGROUND

Immunoassays are used extensively in research to detect the presence of protein in a sample. Traditional immunoassay techniques, however, suffer from many disadvantages. Biological samples typically contain many species, some of which share sequence similarity to the protein of interest. Methods of discriminating between all of the species in a sample can be difficult. It requires tools that can resolve the complex mixture of molecules captured by an antibody, quantify them, and identify them. One factor that reduces the efficacy of immunoassays is their resolution power. For example, in immunoassays using SDS page to separate captured proteins, proteins of very similar size will not be easily distinguishable. Furthermore, antibodies with low sensitivity will be unable to distinguish between closely related proteins or between a protein and its isoformic forms. A proper characterization of the different isoformic forms of a peptide captured in an immunoassay, however, can be necessary to accurately correlate the amount of protein in a sample with a disease state. Another factor that reduces the efficacy of immunoassays is inaccurate information regarding the stability and purity of the reagents. Without the proper tools to accurately quantify the antibody and calibrator used in an immunoassay system, the results of the immunoassay can be compromised.

Accordingly, a need exists for improved methods of discriminating between related proteins in a sample and accurately quantifying the quality and amount of protein present in a sample. This invention meets this and other needs.

### SUMMARY

Methods are described here for discriminating between related proteins and other analytes in a sample and for correlating protein expression with disease states.

In one aspect, a method is described which comprises: capturing polypeptides from a sample. The polypeptides comprise target antigen and at least one modified form of the target antigen. The method further comprises specifically measuring captured target antigen. In some further aspects, the polypeptides are captured with a biospecific capture reagent such as an antibody or a chromatographic adsorbent. In some aspects, the method further comprises measuring at least one modified form of target antigen. In some aspects, the method further comprises capturing and measuring a polypeptide interactor of the target antigen. In some further aspects, the captured polypeptide is measured by mass spectrometry. In some further aspects, the captured polypeptide is measured by affinity mass spectrometry. In some further aspects, the captured polypeptide is measured by SELDI. In some further aspects, the sample is a subject sample and the method further comprises correlating the detected antigen with a clinical parameter in the subject.

In another aspect of the invention, a method is described which comprises capturing at least one modified form of the target antigen polypeptide from a sample. The method further comprises specifically measuring the at least one captured modified form of the target antigen polypeptide. In some further aspects, the polypeptide is captured with a biospecific capture reagent such as an antibody or a chromatographic adsorbent. In some aspects, the method further comprises capturing and measuring a polypeptide interactor of at least one modified form of the target antigen. In some further aspects, the captured polypeptide is measured by mass spectrometry. In other further aspects, the captured polypeptide is measured by affinity mass spectrometry. In other aspects, the captured polypeptide is measured by SELDI. In other further aspects, the sample is a subject sample and the method further comprises correlating the detected modified form of antigen with a clinical parameter in the subject. In some further aspects, the method further comprises capturing and specifically measuring a plurality of modified forms of target antigen from the sample.

In another aspect of the invention, a method is described which comprises providing a learning set. The learning set comprises a plurality of data objects representing subjects. Each data object comprises data representing a specific measurement of target antigen from a subject sample and a clinical parameter of the subject. The method further comprises determining a correlation between the specific measurement of target antigen and the clinical parameters. In some further aspects, providing the learning set comprises the following two steps: (i) capturing target antigen from the sample with an antibody, and (ii) specifically measuring captured target antigen. In one detailed aspect, the captured target antigen is measured by affinity mass spectrometry. In another aspect, the captured target antigen is measured by SELDI.

In another aspect of the invention, a method is described which comprises providing a learning set. The learning set comprising a plurality of data objects representing subjects. The subjects are classified into a plurality of different clinical parameters. Each data object comprises data representing specific measurement of a plurality of polypeptides from a subject sample. The polypeptides are selected from target antigen and at least one modified form of the target antigen. The method further comprises training a learning algorithm with the learning set, thereby generating a classification model. The classification model classifies a data object according to a clinical parameter. In some further aspects, the clinical parameters are selected from presence or absence of disease, risk of disease, stage of disease, response to treatment of disease, and/or class of disease. In some further aspects, the learning set further comprises data representing specific measurement of a polypeptide interactor of the target antigen. In some further aspects, providing the learning set comprises capturing the polypeptides from the sample with an antibody, and specifically measuring captured polypeptides. In one detailed aspect, the captured polypeptides are measured by affinity mass spectrometry. In another aspect, the captured polypeptides are measured by SELDI. In some further aspects, the learning algorithm is unsupervised. In other further aspects, the learning algorithm is supervised and each data object further comprises data representing the clinical parameter of the subject. In some further aspects, the method further comprises using the classification model on subject data from a subject of unknown clinical parameter to classify the subject according to a clinical parameter. In some further aspects, the supervised learning algorithm is selected from linear regression processes, binary decision trees, artificial neural networks, discriminant analyses, logistic classifiers, and support vector classifiers. In other further aspects, the supervised learning algorithm is a recursive partitioning processes.

In another aspect, a method is described for qualifying an immunoassay calibrator for a target antigen immunoassay. The method comprises providing an immunoassay calibrator for a target antigen immunoassay. The calibrator comprises a designated concentration of target antigen. The method further comprises capturing polypeptides from the calibrator with an anti-target antigen antibody and specifically measuring an amount of at least one polypeptide selected from the target antigen and modified form of the target antigen captured by the antibody. The measured amount provides an indication of the quality of the immunoassay calibrator. In a detailed aspect, target antigen is specifically measured. In another detailed aspect, a modified form of the target antigen is specifically measured. In another detailed aspect, target antigen and a modified form of target antigen are specifically measured. In some further aspects, the method comprises determining the amount of target antigen captured as a function of total polypeptide captured by the anti-target antigen antibody. In some further aspects, the anti-target antibody is an antibody used with the immunoassay calibrator in a commercial immunoassay. In a detailed aspect, the amount of target antigen captured is measured by affinity mass spectrometry. In one aspect, the amount of target antigen captures is measured by SELDI.

In another aspect, a method is described for measuring modified forms of an anti-target antigen antibody in an antibody reagent for an immunoassay. In some further aspects, the method comprises measuring unmodified forms of the anti-target antigen antibody in the reagent and further comparing the measurement of unmodified antibody to the measurement of modified forms of the antibody. In one aspect, the anti-target antigen antibody is a monoclonal or a polyclonal antibody. In some further aspects, the method comprise specifically measuring the amount of at least one modified form of the target antigen in the immunoassay calibration sample. In one detailed aspect, the measurements are performed by affinity mass spectrometry. In one aspect, the measurements are performed by SELDI.

In another aspect, a method is described for discovering polypeptides that interact with a target antigen. The method comprises: capturing target antigen from a sample with a biospecific capture reagent, removing molecules that are not bound to the biospecific capture reagent or the target antigen, and measuring molecules bound to the captured target antigen. In one detailed aspect, the molecules are measured by affinity mass spectrometry. In one aspect, the molecules are measured by SELDI.

### DETAILED DESCRIPTION

I. Introduction

The present inventors have discovered that mass spectrometry, and in particular, SELDI, can be used to specifically detect different forms of a protein and protein interactors in a sample. Most immunoassays directed against a particular protein cannot distinguish between different forms of a protein, unless the modification interferes with an epitope recognized by the antibody or antibodies used in the immunoassay. Furthermore, target analytes can be associated with other proteins in a sample with which they interact. Still further, an antibody can bind to molecules in a sample other than the target or modified forms of it. An immunoassay against a target protein typically cannot distinguish these forms or provide detection of proteins that are bound to the target protein. In mass spectrometry, however, analytes are separated by mass and can be distinguished based on their mass signature. Thus, fragments of a protein can be distinguished from a full-length protein. Furthermore, the mass also can indicate the particular location of the fragment within the protein. Other forms of protein decoration, such as phosphorylation, also provide specific mass signatures that can be identified. Accordingly, the present invention provides, inter alia, methods of specifically distinguishing and measuring a target analyte, its modified forms, and biomolecules that interact with these proteins using mass spectrometry.

The present invention also provides, inter alia, methods of discovering diagnostic patterns using a target analyte, modified forms of the target analyte, and biomolecules that interact with these proteins (e.g., discovery phase); and methods of classifying or diagnosing a subject according to a disease based on these proteins.

The present inventors have also discovered that the reagents used in immunoassay systems are, to some extent, degraded. Accordingly, the present invention provides, inter alia, methods of qualifying the reagents, e.g., antibody and calibrator, used in an immunoassay.

The present invention utilizes mass spectrometry, and in particular SELDI, to detect and distinguish a target antigen, its modified forms, biomolecules that interact with the antigen or its modified forms, and immunoassay reagents and to classify disease states in a subject.

II. Specific Detection of a Target Antigen, Modified Forms of the Target Antigen and Biomolecular Interactors with the Target Antigen.

In one aspect, this invention provides methods for specifically measuring a target antigen, modified forms of the target antigen, biomolecular interactors of the target antigen and antibodies that bind to the target antigen. The target antigen, modified forms of the target antigen, biomolecular interactors of the target antigen and antibodies that bind to the target antigen are herein referred to as analytes. Antigens used in all commercial diagnostic tests comprise a polypeptide. Examples of analytes, e.g., antigens, that are subject to the present methods include, but are not limited, GGT, ALT, AST, serum hCG, urine hCG, CKMB, troponin, lipase, amylase, hemoglobin A1C, endocrine such as TSH, prolactin, FSH, LH, and ACTH, CRP, PSA, CA125, CA19-9, CEA, AFP, ferritin, and BNP.

The term “analyte” refers to the substance to be detected that may be present in the sample. The analyte can be any substance for which there exists a naturally occurring specific binding member (such as an antibody or antigen), or for which a specific binding member can be prepared. Thus, an analyte is a substance that can bind to one or more specific binding members in an assay. A biomarker is any analyte that provides information regarding the clinical status of a subject. Modified forms of an analyte, e.g., target antigen, can include, any variant of the analyte. Typically, a variant will have differences in its nucleic acid sequence that results in a recognizable mass shift of the protein level. Differences can include nucleotide polymorphisms, variations in short tandem repeats, variations in allele, or transcriptional varations, e.g., (splice variants).

Specifically measuring an analyte involves detecting the analyte so as to distinguish it from modified forms of the analyte and from biomolecular interactors. The term “measuring” means detecting the presence or absence of an analyte in a sample or quantifying the amount in relative or absolute terms. A relative amount could be, for example, high, medium or low. An absolute amount could reflect the measured strength of a signal or the translation of this signal strength into another quantitative format, such as micrograms/ml. In another aspect, the invention involves comparing the relative ratio of the forms.

A subset of the present methods involve capturing the target antigen, modified forms of the target antigen, biomolecular interactors of the target antigen, and antibodies that bind to the target antigen onto a solid substrate. Typically they will be captured using a biospecific capture reagent against the target antigen such as an antibody and, in particular, an antibody used in an immunoassay. These molecules also can be captured with non-specific methods, such as chromatographic materials. The captured molecules are then specifically detected and distinguished from one another by any appropriate detection means.

III. Detection Methods

The analytes of this invention can be detected by any suitable method. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

A. Detection by Mass Spectrometry

In a preferred embodiment, the analytes of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

1. SELDI

A preferred mass spectrometric technique for use in the invention is “Surface Enhanced Laser Desorption and Ionization” or “SELDI,” as described, for example, in U.S. Pat. No. 5,719,060 and U.S. Pat. No. 6,225,047, each of which is incorporated herein by reference in its entirety and for all purposes. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent can be attached directly to the substrate of the selective surface, or the substrate can have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

Chromatographic adsorbents include those adsorbent materials typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbents” include those molecules that specifically bind to a biomolecule. Biospecific adsorbents generally bind to a target analyte with an affinity of at least 10⁻⁸ M, 10⁻⁹ M, 10⁻¹⁰ M, 10⁻¹¹ M or 10⁻¹² M. Typically they comprise a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Many biospecific capture reagents are known in the art including, for example, antibodies, binding fragments of antibodies (e.g., single chain antibodies, Fab′ fragments, F(ab)′2 fragments, and scFv proteins), affibodies (Affibody, Teknikringen 30, floor 6, Box 700 04, Stockholm SE-10044, Sweden, U.S. Pat. No. 5,831,012, incorporated herein by reference in its entirety and for all purposes)) receptor proteins, nucleic acids, and nucleic acid protein fusions (e.g., from Phylos, Lexington, Mass.). Biospecific adsorbents typically have higher specificity for a target analyte than chromatographic adsorbents. Further examples of adsorbents for use in SELDI can be found in U.S. Pat. No. 6,225,047, incorporated herein by reference in its entirety and for all purposes. Depending on intended use, they also can include receptors and other proteins that specifically bind another biomolecule.

Preferably, the biospecific capture reagent, e.g., biospecific adsorbent, is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact.

In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. In one embodiment, the surfaces of biochips can be derivatized with the capture reagents directed against analytes either in the same location or in physically different addressable locations.

Many protein biochips, adapted for the capture of polypeptides, are described in the art. These include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828, each of which is incorporated herein by reference in its entirety and for all purposes.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with carboimidizole, expoxide) and PG-20 (protein G coupled through carboimidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have have nitriloacetic acid functionalities that adsorb transition metal ions, such as copper, nickel, zinc, and gallium, by chelation. Preactivated ProteinChip arrays have carboimidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); PCT International Publication No. WO 00/66265 (Rich et al., “Probes for a Gas Phase Ion Spectrometer,” Nov. 9, 2000); U.S. Pat. No. 6,555,813 (Beecher et al., “Sample Holder with Hydrophobic Coating for Gas Phase Mass Spectrometer,” Apr. 29, 2003); U.S. Patent Application No. U.S. 2003 0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Provisional Patent Application No. 60/367,837 (Boschetti et al., “Biochips With Surfaces Coated With Polysaccharide-Based Hydrogels,” May 5, 2002) and U.S. Patent Application No. 60/448,467, entitled “Photocrosslinked Hydrogel Surface Coatings” (Huang et al., filed Feb. 21, 2003), each of which is incorporated herein by reference in its entirety and for all purposes.

In general, a probe with an adsorbent surface is contacted with the sample for a period of time sufficient to allow analytes that are present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

The analytes bound to the substrates are detected in a gas phase ion spectrometer such as a time-of-flight mass spectrometer. The analytes are ionized by an ionization source such as a laser, the generated ions are collected by an ion optic assembly, and then a mass analyzer disperses and analyzes the passing ions. The detector then translates information of the detected ions into mass-to-charge ratios. Detection of an analyte typically will involve detection of signal intensity. Thus, both the quantity and mass of the analyte can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003), each of which is incorporated herein by reference in its entirety and for all purposes.

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light (see, U.S. Pat. No. 5,719,060 which is incorporated herein by reference in its entirety and for all purposes). SEPAR and other forms of SELDI are readily adapted to detecting a biomarker or biomarker profile, pursuant to the present invention.

2. Other Mass Spectrometry Methods

In another mass spectrometry method, the analytes can be first captured on a chromatographic resin that binds the target molecules. For example, the resin can be derivatized with an antibody that binds to the target antigen. Alternatively, this method could be preceded by fractionating the sample on an anion exchange resin before application to the cation exchange resin. After elution from the resin, the sample can be analyzed by MALDI, electrospray, or another ionization method for mass spectrometry. In another alternative, one could fractionate on an anion exchange resin and detect by MALDI or electrospray mass spectrometry directly. In yet another method, one could capture the analytes on an immuno-chromatographic resin that comprises antibodies that bind the analytes, wash the resin to remove unbound material, elute the analytes from the resin and detect the eluted analytes by MAIDI, SELDI, electrospray mass spectrometry or another ionization mass spectrometry method.

Despite the numerous mass spectrometry techniques that can be used in the present invention to detect the analytes of interest, SELDI is the preferred technique. SELDI offers several advantages over the other techniques including direct capture on the chip of the analyte, e.g., target antigen, modified forms of the antigen, or biomolecular interactors; improved sensitivity resulting from the ability to wash salts and other signal suppressors off the chip; and direct detection without the need for an intermediate step of elution and re-collection or capture of the analytes.

3. Immunoassay Methods

In another embodiment, the analytes of this invention can be measured by immunoassay. Immunoassay requires biospecific capture reagents, such as antibodies, to capture the analytes. Antibodies can be produced by methods well known in the art, e.g., by immunizing animals with the target antigen or protein. Analytes can be isolated from samples based on their binding characteristics. Alternatively, if the amino acid sequence of a polypeptide analyte is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. In the SELDI-based immunoassay, a biospecific capture reagent for the analyte is attached to the surface of an MS probe, such as a pre-activated ProteinChip array. The analyte can then be specifically captured on the biochip through this reagent, and the captured analyte can be detected by mass spectrometry.

IV. General Protocol for SELDI Detection of a Target Antigen, Modified Forms of the Antigen and Biomolecular Interactors

One preferred protocol for the detection of the analytes of this invention is as follows. The biological sample to be tested as used herein is a sample of biological tissue or fluid and includes human and animal body fluid such as whole blood, plasma, white blood cells, cerebrospinal fluid, urine, semen, vaginal secretions, lymphatic fluid, and various external secretions of the respiratory, intestinal and genitourinary tracts, tears, saliva, milk, ductal lavage, seminal plasma, tissue biopsy, fixed tissue specimens, fixed cell specimens, cell extracts and cell culture supernatents and derivatives of these, e.g., blood or a blood derivative such as serum, preferably is subject to pre-fractionation before SELDI analysis. This simplifies the sample and improves sensitivity. A preferred method of pre-fractionation involves contacting the sample with an anion exchange chromatographic material, such as Q HyperD (BioSepra, SA). The bound materials are then subject to stepwise pH elution using buffers at pH 9, pH 7, pH 5 and pH 4. Various fractions containing the analtyes are collected.

The sample to be tested (preferably pre-fractionated) is then contacted with an affinity capture probe comprising an antibody that binds to the antigen of interest, e.g., a pre-activated PS10 or PS20 ProteinChip array (Ciphergen Biosystems, Inc.). The probe is washed with a buffer that will retain the antigen, modified forms of the antigen, biomolecular interactors of the antigen and/or antibodies that bind to the target antigen while washing away unbound molecules. A suitable wash for these molecules is the buffer identified in the Example. The analytes are detected by laser desorption/ionization mass spectrometry.

V. Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample. Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data can be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

VI. Correlation of a Specific Measurement of Target Antigen, Different Forms of Target Antigen, and Interactors with Specific Clinical Parameters

A. Clinical Diagnostics

A principle of diagnostic testing is the correlation of the results of a procedure (e.g., blood test, urine test, CSF, test, sputum test, tissue biopsy, radiologic examination, measurement of one or more biomarkers, and the like) with particular clinical parameters. The correlation necessarily involves a comparison between two or more groups distinguished by the clinical parameter. A clinical parameter could be, for example, presence or absence of disease, risk of disease, stage of disease, severity of disease, class of disease or response to treatment of disease. Accordingly, the diagnostician uses this correlation to qualify the status of a subject with respect to the clinical parameter. That is, the diagnostician uses the results of a procedure on a subject to classify or diagnose a subject status with respect to a clinical parameter, the confidence of the diagnosis/classification being related to the classifying or splitting power of the signs or symptoms used in the test.

Biomarkers having the most diagnostic utility show a statistical difference in different clinical parameters of at least p≦0.05, p≦10⁻², p≦10⁻³, p≦10⁻⁴ or p≦10⁻⁵. Diagnostic tests that use these biomarkers alone or in combination show a sensitivity and specificity of at least 75%, at least 80%, at least 85%, at least 90%, at least 95%, at least 98% and about 100%.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives that test as positive. Negative predictive value is the percentage of actual negatives that test as negative.

In certain conditions, elevated levels of a target antigen will correlate with a disease state. The diagnostician can use a measurement of the target antigen to qualify the disease status of a subject. For example, a doctor can use the amount of target antigen in a patient blood sample to diagnose the presence or absence of disease.

A typical immunoassay does not distinguish between the target antigen and modified forms of the target antigen captured by the antibody, and also does not detect protein interactors. Therefore, the typical immunoassay results in the correlation of all molecular forms of the antigen together with the clinical parameter of interest. However, by specifically distinguishing the measurements of antigen, its various forms and interactors, this invention allows the specific correlation of these analytes with the clinical parameter. Specific correlation of particular analytes in a sample provides greater specificity and sensitivity in diagnosis.

B. Use of Specific Measurement of Target Antigen and Its Forms in Determining Clinical Status of Patients

Accordingly, in one aspect this invention provides diagnostic, prognostic and theranostic methods using the specific measurement of at least one biomarker selected from a target antigen, modified forms of the target antigen or biomolecular interactors of the target antigen and antibodies that bind to the target antigen. The methods involve first providing a specific measurement of the form of target antigen by any method, and then correlating the measurement with the clinical parameter of interest. By correlating the measurement, one is able to qualify the subject status with respect to the particular clinical parameter in question. Based on this correlation, further procedures can be indicated, including additional diagnostic tests or therapeutic procedures or regimens. Each of the biomarkers of this invention can be individually correlated with disease.

Any form of target antigen or protein interactor, individually, is useful in aiding in the determination of disease status. First, the selected biomarker is specifically measured in a subject sample using the methods described herein, e.g., capture on a SELDI biochip followed by detection by mass spectrometry. Then, the measurement is compared with a diagnostic amount or cutoff that distinguishes one diagnostic parameter from another. The diagnostic amount represents a measured amount of a biomarker above which or below which a subject is classified as having a particular clinical parameter. For example, if the biomarker is up-regulated compared to normal in clinical parameter, then a measured amount above the diagnostic cutoff provides a diagnosis of clinical parameter. Alternatively, if the biomarker is down-regulated in acute coronary syndrome, then a measured amount below the diagnostic cutoff provides a diagnosis of acute coronary syndrome. As is well understood in the art, by adjusting the particular diagnostic cutoff used in an assay one can increase sensitivity or specificity of the diagnostic assay depending on the preference of the diagnostician.

In some embodiments, the mere presence or absence of a biomarker, without quantifying the amount of the biomarker, is useful and can be correlated with a probable diagnosis of disease. Thus, a detected presence or absence, respectively, of these markers in a subject being tested indicates that the subject has a higher probability of having a specific disease.

While individual biomarkers are useful diagnostic markers, it has been found that a combination of biomarkers can provide greater predictive value of a particular status than single markers alone. Specifically, the detection of a plurality of markers in a sample can increase the percentage of true positive and true negative diagnoses and decreases the percentage of false positive or false negative diagnoses. Thus, in one embodiment, one measures the relative ratio of various forms of target antigen, modified forms of target antigen or biomolecular interactors with the target antigen.

In certain embodiments of the methods of qualifying a disease status, the methods further comprise managing subject treatment based on the status. Such management describes the actions of the physician or clinician subsequent to determining acute coronary syndrome status. For example, if a physician makes a diagnosis of acute coronary syndrome, then a certain regime of treatment, such as medical intervention (e.g. statins, beta blocker, glycoprotein IIb/IIIa inhibitor) or invasive intervention (e.g., revascularization) might follow. The specific complement of biomarkers and their interactors can predict the optimal course of treatment. Alternatively, a diagnosis of non-acute coronary syndrome might be followed with no treatment. If the diagnostic test gives an inconclusive result on acute coronary syndrome status, further tests may be called for.

VII. Pattern Discovery

While single target analytes have traditionally been used as correlates of clinical parameters, such as presence or absence of disease, scientists and physicians have taken increasing interest in the use of multiple makers. The discovery of patterns of molecules that can be correlated with a clinical parameter involves the multivariate analysis of measurements of a plurality of molecules, such as proteins, in a sample.

Accordingly, in one aspect this invention provides a method for discovering patterns of proteins including a target antigen, modified forms of the antigen or biomolecules that interact with these, which patterns correlate with a clinical parameter of interest.

While one can identify patterns by visual inspection of the data, in the case of large amounts of data it is preferred to subject the data to a learning algorithm executed by a computer. This method involves training a learning algorithm with a learning set of data that includes measurements of the aforementioned molecules and generating a classification algorithm that can classify an unknown sample into a class represented by clinical parameter.

In this case, pattern analysis involves training a learning algorithm with a learning set of data that includes measurements of the aforementioned molecules and generating a classification algorithm that can classify an unknown sample into a class represented by clinical parameter.

The method involves, first, providing a learning set of data. The learning set includes data objects. Each data object represents a subject for which clinical data has been developed. In on embodiment, clinical data included in the data object includes the specific measurements of target antigen, modified forms of target antigen and biomolecular interactors and antibodies that with these. Each subject is classified into one of at least two different clinical parameter classes. For example, the clinical parameters could include presence or absence of disease, risk of disease, stage of disease, response to treatment of disease or class, prognosis or kind of disease.

In a preferred embodiment, the learning set will be in the form of a table in which, for example, each row is data object representing a sample. The columns contain information identifying the subject, data providing the specific measurements of each of the molecules measured and optionally identifying the clinical parameter associated with the subject.

The learning set is then used to train a classification algorithm. Classification models can be formed using any suitable statistical classification (or “learning”) method that attempts to segregate bodies of data into classes based on objective parameters present in the data. Classification methods can be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, each data object includes data indicating the clinical parameter class to which the subject belongs. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines). A preferred supervised classification method is a recursive partitioning process. Recursive partitioning processes use recursive partitioning trees to classify spectra derived from unknown samples.

In other embodiments, the classification models that are created can be formed using unsupervised learning methods. Unsupervised classification attempts to learn classifications based on similarities in the training data set. In this case, the data representing the class to which the subject belongs is not included in the data object representing that subject, or such data is not used in the analysis. Unsupervised learning methods include cluster analyses. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application 2002 0193950 A1 (Gavin et al., “Method or analyzing mass spectra”), U.S. Patent Application 2003 0004402 A1 (Hitt et al., “Process for discriminating between biological states based on hidden patterns from biological data”), and U.S. Patent Application 2003 0055615 A1 (Zhang and Zhang, “Systems and methods for processing biological expression data”), each of which is incorporated herein by reference in its entirety and for all purposes.

Thus trained, learning algorithm will generate a classification model that classifies a sample into one of the classification groups. The classification model usually involves a subset of all the markers included in the learning set. The classification model can be used to classify an unknown sample into one of the groups.

A learning algorithm, such as CART, can detect many different patterns in the learning set that are useful for classifying a sample into one of the groups. These patterns most likely will differ based not only on the specific markers employed in the classification algorithm, but also in the specific function of amount of the molecule in the sample (e.g., the cut-off value). However, it also is typical that among many patterns generated, certain of the proteins recur frequently, indicating that they are particularly useful as “splitters” in classification algorithms to classify a sample into one group or another.

A. Clinical Assay Phase

Once the learning algorithm has generated a classification algorithm, the classification algorithm can be used in a clinical setting to classify a subject sample according to the clinical parameter that is the subject of the test. The clinical assay phase can include the following steps: (1) collecting a sample from a subject to be tested; (2) measuring the particular analytes that form the classification pattern; (3) comparing this data to the diagnostic classification pattern; e.g., submitting the data to the classification algorithm and (4) assigning the sample to one of the groups based on the pattern, e.g., based on the result of application of the classification algorithm.

The classification algorithm is keyed to the particular assay conditions under which it was developed. That is to say, in order to generate a useful result from a clinical test, it must be performed according to the same protocol as used to generate the data which was submitted to the learning algorithm. Changes in parameters such as sample source and measurement assay conditions will most likely result in data that cannot be properly interpreted by the classification algorithm. This is because the classification algorithm is likely to key on subtle relationships between particular molecules (the “pattern”). These relationships will probably be disrupted if different clinical assay conditions are used. For example, the use of a different wash buffer on a chip might alter the relative amount of two proteins retained on the chip. If this relative amount is used in the classification algorithm, then changing it by changing the assay conditions will also change the result of the test.

As stated, the analytes used in the classification algorithm will generally be a subset of the analytes measured in the discovery phase. Accordingly, in carrying out a clinical diagnostic assay keyed to the proteins in the classification algorithm, one need only specifically measure those key analytes. These measurements then can be submitted to the classification algorithm for analysis. Alternatively, measurements can be obtained for a broad spectrum of analytes. Absence of changes for subsets of these proteins can, in fact, contribute to the specificity of the diagnosis.

Upon submission of the specific measurements called for the classification algorithm, the algorithm will generate a classification of the sample into one of the clinical parameters to which the test is directed. This result can aid the diagnostician by indicating that a particular clinical parameter is present, or by ruling out certain clinical parameters.

One can then manage subject treatment based on the result of the diagnostic test. For example, if disease is present, a certain course of treatment can be prescribed. Alternatively, if the result is ambiguous, further texts can be ordered. Tests can be performed sequentially, to provide monitoring of a patient for the progression of the disease or the effect of treatment or the status of recovery.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives that test as positive. Negative predictive value is the percentage of actual negatives that test as negative.

VIII. Determining the Quality of an Immunoassay Calibrator

Calibration of an immunoassay is important for ensuring the quality of results generated in the immunoassay. Calibration generally involves the use of an immunoassay calibrator that contains the target analyte in a prescribed amount or concentration. The signal produced by the calibrator in an immunoassay is correlated to the amount of target analyte in the calibrator. This calibration, in turn, is used to correlate the amount of signal measured in a test sample with an amount of target analyte in the test sample. However, the signal generated by the calibrator may not represent the true amount of analyte in the calibrator if, for example, the target analyte in the calibrator is degraded or otherwise modified so as to corrupt the signal.

Accordingly, this invention provides methods for determining the quality of an immunoassay calibrator. The method involves capturing molecules from a immunoassay calibrator used in an immunoassay against a target antigen with an antibody that captures the target antigen and specifically measuring the amount of target antigen or one or more modified forms of target antigen captured by the antibody. Alternatively, the immunoassay could be directed to measuring a particular modified form of target antigen and involve the use of antibodies against this form and a calibrator that included this form.

IX. Determining the Quality of an Antibody in an Antibody Reagent Used in an Immunoassay

Immunoassays typically involve the use an immunoassay reagent that comprises an antibody directed against the target analyte. The accuracy of such assays depends upon the integrity and purity of the antibody in the immunoassay reagent. The presence of contaminants in an antibody reagent can interfere with an accurate measurement of the amount of antibody in the antibody reagent. Accordingly, the present invention provides methods for determining the quality of an anti-target antigen antibody (e.g., antibody directed against the target antigen) used in an immunoassay reagent by specifically detecting modified forms and/or un-modified forms of the antibody, e.g., degraded forms, in the reagent.

In one version of the method, an antibody used in an immunoassay, in particular a commercial immunoassay, is examined by mass spectrometry. This analysis can indicate what portion of the antibody reagent is whole and what part is degraded. For example, the immunoglobulin may be degraded into heavy chains and light chains. Also, the immunoglobulin may be degraded into fragments of the heavy and light chains. Because mass spectrometry can distinguish intact immunoglobulin and degraded versions of it based on mass differences, the immunoglobulin reagent can thereby be qualified.

In another version of the method, the antibody is coupled to the surface of a SELDI probe and used to capture target antigen from a sample or from a target antigen calibrant for an immunoassay. This method can detect the absolute amount of intact target antigen captured, as well as the relative amount of intact target antigen to other molecules. The absolute quantity of an analyte as measured by an immunoassay is dependent on the quality of the reagents used to measure the analyte, as well as the quality of the reagents used to generate the standard curve (i.e. the calibrators). If the antibody is not specific for the intended analyte, it can give false elevated levels. If the calibrator is impure, the calibration curve will be inaccurate. The inaccurate quantitation of an analyte can lead to the generation of incorrect conclusions regarding the optimal cutoffs for making medical decisions and can lead to the incorrect quantitation in individuals, leading to suboptimal management.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention

1. A method comprising:
capturing polypeptides from a sample, wherein the polypeptides comprise target antigen and at least one modified form of target antigen; and
specifically measuring captured target antigen.

2. The method of claim 1 wherein the polypeptides are captured with an antibody.

3. The method of claim 1 wherein the polypeptides are captured with a chromatographic sorbent.

4. The method of claim 1 further comprising specifically measuring at least one modified form of target antigen.

5. The method of claim 1 further comprising capturing and measuring a polypeptide interactor of the target antigen.

6. The method of claim 1 wherein the captured polypeptides are measured by mass spectrometry.

7. The method of claim 1 wherein the captured polypeptides are measured by affinity mass spectrometry.

8. The method of claim 1 wherein the polypeptides are measured by SELDI.

9. The method of claim 1 wherein the sample is a subject sample and the method further comprises:
(c) correlating the detected target antigen with a clinical parameter in the subject.

10. The method of claim 9 wherein the clinical parameter is presence or absence of a disease associated with the target antigen.

11. A method comprising:
capturing at least one modified form of the target antigen polypeptide from a sample; and
specifically measuring the at least one captured modified form of the target antigen polypeptide.

12-19. (canceled)

20. A method comprising:
providing a learning set comprising a plurality of data objects representing subjects, wherein each data object comprises data representing a specific measurement of target antigen from a subject sample and a clinical parameter of the subject; and
determining a correlation between specific measurement of target antigen and the clinical parameters.

21-23. (canceled)

24. A method comprising:
providing a learning set comprising a plurality of data objects representing subjects, wherein the subjects are classified into a plurality of different clinical parameters and wherein each data object comprises data representing specific measurement of a plurality of polypeptides from a subject sample wherein the polypeptides are selected from target antigen and at least one modified form of target antigen; and
training a learning algorithm with the learning set, thereby generating a classification model, wherein the classification model classifies a data object according to clinical parameter.

25-34. (canceled)

35. A method for qualifying an immunoassay calibrator for an target antigen immunoassay comprising:
providing an immunoassay calibrator for an target antigen immunoassay, wherein the calibrator comprises a designated concentration of target antigen;
capturing polypeptides from the calibrator with an anti-target antigen antibody; and
specifically measuring an amount of at least one polypeptide selected from target antigen and modified form of the target antigen captured by the antibody, whereby the measured amount provides an indication of the quality of the immunoassay calibrator.

36-42. (canceled)

43. A method comprising measuring modified forms of an anti-target antigen antibody in an antibody reagent for a target antigen immunoassay.

44-47. (canceled)

48. A method for discovering polypeptides that interact with target antigen comprising:
capturing target antigen from a sample with a biospecific capture reagent;
removing molecules that are not bound to the biospecific capture reagent or the target antigen; and
measuring molecules bound to the captured target antigen.

49-50. (canceled)
